(19) United States
(12) Patent Application Publication
(10) Pub. No.: US 2014/0348931 A1
(43) Pub. Date: Nov. 27, 2014

(54) SUSTAINED-RELEASE TOPIRAMATE FORMULATIONS

(71) Applicant: CADILA HEALTHCARE LIMITED, AHMEDABAD (IN)

(72) Inventors: SUSHRUT KRISHNAJI KULKARNI, AHMEDABAD (IN); AJAYKUMAR HANDA, AHMEDABAD (IN); AMIT OM PRAKASH GUPTA, AHMEDABAD (IN); PRUDHVI RAJU DANTULURI, AHMEDABAD (IN)

(73) Assignee: CADILA HEALTHCARE LIMITED, AHMEDABAD (IN)

(21) Appl. No.: 14/287,787

(22) Filed: May 27, 2014

(30) Foreign Application Priority Data
May 27, 2013 (IN) 1868/MUM/2013

(51) Int. Cl. A61K 9/16; A61K 31/357

(52) U.S. Cl. CPC A61K 9/1676; A61K 31/357; A61K 9/1682
USPC 424/490; 514/454; 427/2.21

(57) ABSTRACT

The present invention relates to a sustained release pharmaceutical formulation comprising topiramate and one or more pharmaceutically acceptable excipients, wherein the formulation comprises two extended release (XR) components or a combination of an extended release (XR) component and immediate release (IR) component, wherein at least one component is in a matrix form. It also relates to method of preparing such formulations and using those formulations in the treatment of neurological and/or psychiatric condition.

### FIELD OF THE INVENTION

The present invention relates to a sustained release pharmaceutical formulation comprising topiramate and one or more pharmaceutically acceptable excipients, wherein the formulation comprises two extended release (XR) components or a combination of an extended release (XR) component and immediate release (IR) component, wherein at least one component is in a matrix form. It also relates to method of preparing such formulations and using those formulations in the treatment of neurological and/or psychiatric condition.

### BACKGROUND OF THE INVENTION

Topiramate is designated chemically as 2, 3: 4, 5 Di-O-isopropylidene-β-D-fructopyranose sulfamate and has the following structural formula:

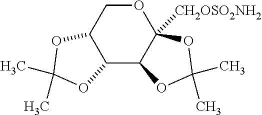

Topiramate is a sulfamate substituted monosaccharide which under the tradename TOPAMAX® (Ortho-McNeil Pharmaceutical, Inc., Raritan, N.J., U.S.A.) has been approved for use as an antiepileptic agent, as an adjuvant therapy for patients with partial onset seizures or primary generalized tonic-clonic seizures, and for the prevention of migraine (Physician's Desk Reference, 60^{th} ed., 2538-2447 (2006); U.S. Pat. No. 4,513,006).

For the treatment of epilepsy, the recommended dose of TOPAMAX® is 400 mg/day in one or multiple doses (Physician's Desk Reference, 60^{th} ed., 2538-2447 (2006)). For the treatment of epilepsy in adults, treatment is initiated with a dose of 25-50 mg/day, with the dose being titrated in increments of 25-50 mg at weekly intervals to the recommended or effective dose. Topamax® is an immediate release formulation. Adverse effects associated with the administration of Topamax® include, but are not limited to, somnolence, dizziness, ataxia, speech disorders and related speech problems, psychomotor slowing, abnormal vision, difficulty with memory, paresthesia, diplopia, renal calculi (kidney stones), hepatic failure, pancreatitis, renal tubular acidosis, acute myopia and secondary angle closure glaucoma (Physician's Desk Reference, 10^{th} ed., 2538-2447 (2006)).

Hence, though topiramate has a relatively long half-life of 21 hours in vivo, it has not been prescribed (or formulated) as a single, daily-dose, in part due to severe side-effects that often result with peak plasma levels of the drug when taken in high doses. Instead, Topamax® is typically taken in multiple, “divided” doses, usually twice-daily (“BID”). However, administration of the medicament in this manner is cumbersome and patients can forget to take their medication in a timely manner. Moreover, each administration of a dose is associated with a peak in plasma concentrations of the drug, and the fluctuations associated with the peaks and valleys of blood plasma levels of the drug are undesirable. Therefore, there is a need for a formulation of topiramate, which reduces or eliminates the side effects associated with peaking and fluctuating plasma levels of the drug and preferably may be administered in a once-daily regimen.

PCT application No. WO 2008-070670 describes a novel enhanced immediate release formulation of topiramate for oral administration to a mammalian subject, wherein at least 80% of the active compound is dissolved in a time period of not more than 30 minutes. The formulation comprises topiramate as an active ingredient and at least one complexing agent. The application describes a highly soluble complex of topiramate with a cyclodextrin which is selected from a group consisting of hydroxypropyl-β-cyclodextrin, β-cyclodextrin, γ-cyclodextrin, and α-cyclodextrin, or its derivative.

The sustained release formulation of topiramate with an effective single daily dose regimen enables it to improve patient compliance and may also reduce some of the side effects of topiramate associated with existing higher daily doses of immediate release topiramate formulations.

A more soluble and bioavailable form of topiramate has been described in PCT application No. WO 2008-061226 for once-daily sustained-release dosage form of topiramate or salts thereof wherein the formulation comprises an enhanced immediate release coated bead population in addition to two extended release (XR) coated bead populations, wherein each XR component comprises a release controlling coating which is specific for every population of beads and determines the rate of release of topiramate from the given bead population.

However, there is still a need for an alternate sustained-release topiramate formulation which could ease and simplify the overall formulation process.

The instant invention addresses these and other needs by providing alternate modified formulations of topiramate characterized by a sustained release of an active ingredient. This invention additionally provides an effective, once-daily dosage form of topiramate or salts thereof, which not only enables an effective single daily dose regimen to improve patient compliance but may also reduce some of the side effects of topiramate compared to the current or higher daily doses of immediate release topiramate formulations.

### SUMMARY OF THE INVENTION

In one general aspect, there is provided a sustained release formulation of topiramate comprising two extended release components, a first extended release component (XR1) and a second extended release component (XR2), wherein topiramate is present in both the components and at least one of the components is present in a matrix form.

In another general aspect, there is provided a sustained release formulation of topiramate, wherein the XR1 component is a matrix based formulation and the XR2 component a coated bead formulation.

In another general aspect, there is provided a sustained release formulation of topiramate, wherein the XR1 component comprises up to 50% by wt. of the total amount of the topiramate in the formulation and the XR2 component comprises at least 50% by wt. of the total amount of the topiramate in the formulation.

Embodiments of the pharmaceutical formulation may include one or more of the following features. For example, the pharmaceutical formulation may further include one or more pharmaceutically acceptable excipients. The pharmaceutically acceptable excipients may include one or more fillers, disintegrants, binders, lubricants, glidants, anti-tacking agents, plasticizers, and the like.

In another general aspect, there is provided a formulation which can be in the form of a tablet, a capsule, a caplet, a pouch, pellets, beads, sprinkles, granules or powder.

In another general aspect, there is provided a sustained release formulation of topiramate wherein the formulation exhibits no significant difference in both rate and extent of absorption of topiramate as compared to extended release formulation of topiramate marketed under the trade name Trokendi®.

In yet another general aspect, there is provided a sustained release formulation of topiramate, wherein the formulation provides a mean AUC and a mean Cₘₐₓ of plasma topiramate in both fed and fasted states within 80% to 125% of mean AUC and mean Cₘₐₓ of plasma topiramate provided by a topiramate extended release reference standard upon single dose administration to a population of human subjects respectively.

In another general aspect, there is provided a sustained release formulation of topiramate comprising an immediate release component (IR) comprising topiramate and an extended release component (XR) comprising topiramate, wherein topiramate is present in both the components and at least one of the components is in a matrix form.

In another general aspect, there is provided a sustained release formulation of topiramate, wherein the extended release component is prepared by a process comprising:
(i) coating a first layer of topiramate on inert carrier particles;
(ii) coating the particles with a second layer comprising one or more controlled release agents to obtain pellets; and
(iii) optionally, employing a barrier layer between the first and the second layer.

In yet another general aspect, there is provided a sustained release formulation of topiramate, wherein the extended release component is prepared by a process comprising:
(i) mixing and/or granulating topiramate, one or more controlled release agents and one or more pharmaceutically acceptable excipients to form a wet mass;
(ii) extruding the wet mass of step (i);
(iii) spheronizing the product of step (ii) to form pellets; and
(iv) processing the pellets to form the extended release component of the final formulation.

In another general aspect, there is provided a sustained release formulation of topiramate, wherein the extended release component is prepared by a process comprising:
(i) mixing and/or granulating topiramate, one or more controlled release agents and one or more pharmaceutically acceptable excipients to form a wet mass; and
(ii) drying, lubricating and compressing the obtained mass to form the extended release component of the final formulation.

Embodiments of the pharmaceutical formulation may include one or more of the following features. For example, the pharmaceutical formulation may further include one or more pharmaceutically acceptable excipients. The pharmaceutically acceptable excipients may include one or more fillers, disintegrants, binders, lubricants, glidants, anti-tacking agents, plasticizers, and the like.

In another general aspect, there is provided a method of treatment of a neurological and/or psychiatric condition, comprising orally administering to the subject a therapeutically effective amount of the sustained release formulation of topiramate, wherein said condition is selected from a group consisting of epilepsy, migraine, essential tremor, restless limb syndrome, cluster headaches, neuralgia, neuropathic pain, tourrette's syndrome, infantile spasms, bipolar disorder, dementia, depression, psychosis, mania, anxiety, schizophrenia, obsessive-compulsive disorder, post-traumatic stress disorder, attention deficit hyperactivity disorder, impulse control disorders, border line personality disorder, addiction, autism, chronic neurodegenerative disorders, acute neurodegeneration, amyotrophic lateral sclerosis.

The details of one or more embodiments of the invention are set forth in the description below. Other features of the invention will be apparent from the description.

### DETAILED DESCRIPTION OF THE INVENTION

The present invention provides a sustained release formulation of topiramate for oral administration comprising topiramate as an active ingredient, wherein topiramate is released from the formulation at a sustained rate along a pre-determined release profile, and wherein the sustained release formulation comprises two extended release components or a combination of an extended release (XR) component and an immediate release (IR) component, wherein at least one component is in a matrix form.

It has now surprisingly been found that a topiramate formulation comprising two extended release components or a combination of an extended release component (XR) and a matrix based immediate release (IR) component can be provided, which releases at least 80% of the topiramate in a continuous manner in less than or equal to about 12 hours.

For the purposes of this application, the term “topiramate” includes topiramate or any pharmaceutically acceptable salts or derivatives thereof, including polymorphs, hydrates, solvates or amorphous forms.

An “immediate release formulation” refers to a formulation that releases 70-80% of the pharmaceutically active ingredient in less than or equal to about 1 hour.

The term “extended release” as used herein can be used synonymously with sustained release, controlled release, modified release and delayed release. The first extended release component is termed as XR1 and the second extended release component is termed as XR2.

The term “sustained release” is defined herein as release of a pharmaceutically active agent in a continuous manner over a prolonged period of time.

By “prolonged period of time” it is meant a continuous period of time of greater than about 1 hour, preferably, greater than about 4 hours, more preferably, greater than about 8 hours, more preferably greater than about 12 hours, more preferably still, greater than about 16 hours up to more than about 24 hours.

The term “beads”, as used herein, includes, without any limitations on the nature and size thereof, any particles, spheres, beads, granules, pellets, particulates or any structural units that may be incorporated into an oral dosage form.

In one embodiment, the extended release (XR) component is contained in a matrix comprising one or more controlled release agent.

In another embodiment, the extended release (XR) component is contained in a population of beads coated with a coating that modifies and controls the release of topiramate from the coated beads (release controlling coating). The release controlling coating is specific for every population of beads and determines the rate of release of topiramate from the given coated bead population.

In yet another embodiment, the immediate release (IR) component is contained in a matrix comprising one or more pharmaceutically acceptable excipients.

As used herein, unless otherwise noted, “rate of release” or “release rate” of a drug refers to the quantity of drug released from a dosage form per unit time, e.g., milligrams of drug released per hour (mg/hr) or a percentage of a total drug dose released per hour. Drug release rates for dosage forms are typically measured as an in vitro rate of drug release i.e., a quantity of drug released from the dosage form per unit time measured under appropriate conditions and in a suitable fluid.

The release rates referred to herein are determined by placing a dosage form to be tested in a medium in an appropriate dissolution bath. Aliquots of the medium, collected at pre-set intervals, are then injected into a chromatographic system fitted with an appropriate detector to quantify the amounts of drug released during the testing intervals.

Another embodiment discloses a sustained release formulation comprising a first extended release component (XR1) and a second extended release component (XR2), wherein the XR1 component comprises up to 50% by weight of the total amount of topiramate in the formulation, more preferably up to 35% by weight and the XR2 component comprises at least 50% by weight of the total amount of topiramate in the formulation, more preferably at least 65% by weight.

Another embodiment discloses a sustained release formulation comprising a first extended release component (XR1) and a second extended release component (XR2), wherein the XR1 component releases about 75%, more preferably about 80% of the topiramate in vitro in less than or equal to about 3 hours and about 90%, more preferably about 97% of the topiramate in vitro in less than or equal to about 6 hours.

Another embodiment discloses a sustained release formulation comprising a first extended release component (XR1) and a second extended release component (XR2), wherein the XR2 component releases about 80%, more preferably about 85% of the topiramate in vitro in less than or equal to about 6 hours and releases about 90% of the topiramate in vitro in less than or equal to about 12 hours.

Another embodiment discloses a sustained release formulation comprising a matrix based immediate release (IR) component and an extended release (XR) component, wherein the IR component comprises up to 30% by weight of the total amount of topiramate in the formulation, more preferably up to 25% by weight and the XR2 component comprises at least 70% by weight of the total amount of topiramate in the formulation, more preferably at least 75% by weight.

Another embodiment discloses a sustained release formulation of topiramate, wherein the formulation releases about 30% of topiramate in about less than or equal to about 1 hour, releases about 35 to about 75% of topiramate in less than or equal to about 3 hours, releases more than about 80% topiramate in less than or equal to about 12 hours.

The composition of the present invention may further comprise other pharmaceutically active agents suitable for use in combination with topiramate for treatment or prevention of a pathological condition. The additional pharmaceutically active agents, without limitation, may be represented by analgesic and anti-inflammatory compounds such as COX-2 inhibitors, nonsteroidal anti-inflammatory drugs (NSAIDs), narcotic drugs such as opiates and morphinomimetics, synthetic drugs with narcotic properties such as tramadol; anticonvulsants such as valproic acid or its derivatives, carbamazepine, oxcarbazepine, gabapentin, and lamotrigine; anorectics or anti-obesity agents such as sibutramine or other, orlistat or other pancreatic lipase inhibitors, diethylpropion, fluoxetine, bupropion, amphetamine, methamphetamine, sertraline, zonisamide, and metformin, as well as medications associated with weight-gain, such as sulfonylurea derivatives, insulin, and thiazolidinediones whose weight-gain effect is tempered by topiramate; anti-hypertensive agents such as diuretics, anti-adrenergics, calcium channel blockers, ACE inhibitors, angiotensin II receptor antagonists, aldosterone antagonists, vasodilators, centrally acting adrenergic drugs, and adrenergic neuron blockers; mood stabilizers such as various forms/salts of lithium, Omega-3 fatty acids and others known in the art, drugs for treatment or prevention of migraines, such as ergot derivatives or triptans, or any other pharmaceutical or nutraceutical ingredient that can be safely and beneficially combined with topiramate.

The sustained release formulation may be in the form of a tablet, a capsule, a caplet, a pouch, sprinkles, pellets, granules, or powder.

The inert carriers useful in the present invention may be selected from, but are not limited to a group consisting of cellulose spheres, silicon dioxide and starch or sugar spheres. The inert carrier is present in an amount from about 10% to about 99% by weight, and preferably in an amount from about 40% to about 97% by weight of the component.

The one or more controlled release agent of the present invention may be selected from, but are not limited to, hydrophilic or hydrophobic materials or combinations thereof.

Suitable hydrophilic materials may include one or more of cellulose derivatives, polysaccharides, a polyacrylate, polyvinyl alcohol or polyvinyl pyrrolidone, carbopols, polyethylene oxides, magnesium aluminum silicate, modified starch derivatives or a derivative of such hydrophilic polymers or combinations thereof.

Suitable cellulose derivatives may include one or more of methylcellulose, ethyl cellulose, hydroxymethyl cellulose, different viscosity grades of hydroxypropyl methylcellulose, hydroxy propyl cellulose, hydroxyethylcellulose, carboxymethyl cellulose or a combination thereof.

The polysaccharides suitable for the purposes of the present invention may include one or more of gums both natural and modified (semi-synthetic) like alginates, Karaya, Guar, Locust bean, xanthan gum, gellan gum, welan gum, rhamsan gum and dextran.

Suitable hydrophobic material may include one or more of waxes, ethylcellulose, copolymer of acrylic acid and methacrylic acid esters, polyethylene, polyamide, polyvinyl acetate, glycerol monostearate, stearylalcohol, glyceryl behenate or mixtures thereof.

Suitable wax material may include one or more of an amorphous wax, an anionic wax, an anionic emulsifying wax, a bleached wax, a carnauba wax, a cetyl esters wax, a beeswax, hydrogenated castor oil, hydrogenated vegetable oil, a cationic emulsifying wax, a cetrimide emulsifying wax, an emulsifying wax, glyceryl behenate, a microcrystalline wax, a nonionic wax, a nonionic emulsifying wax, a paraffin, a petroleum wax, a spermaceti wax, a white wax, a yellow wax, and combinations comprising one or more of the foregoing waxes. These and other suitable waxes are known to those having skill in the art.

The coating for extended release component comprises a controlled release agent and, optionally, a pore former and other excipients. The coating may comprise cellulosic polymers, such as ethylcellulose, methylcellulose, hydroxypropyl cellulose, hydroxypropyl methylcellulose, hydroxypropyl methylcellulose phthalate, cellulose acetate, and cellulose acetate phthalate; polyvinyl alcohol; acrylic polymers such as polyacrylates, polymethacrylates and copolymers thereof, and other water-based or solvent-based coating materials.

The pharmaceutically acceptable excipients of the present invention may further include one or more fillers, disintegrants, binders, lubricants, glidants, anti-tacking agents, plasticizers, and the like.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like.

Suitable disintegrants may include one or more of croscarmellose sodium, sodium starch glycolate, pregelatinized starch, sodium carboxymethyl cellulose, cross-linked polyvinylpyrrolidone and the like.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinylpyrrolidone, pregelatinized starch, sodium alginate, gums, synthetic resins and the like. The binder may be present in the IR or XR formulation in an amount of from about 0.1% to about 15% by weight, and preferably of from about 0.2% to about 10% by weight.

Suitable pore forming agents may include glucose, fructose, mannitol, mannose, galactose, sorbitol, pullulan, dextran, hydroxyalkylcelluloses, carboxyalkylcelluloses, hydroxypropyl methylcellulose, cellulose ethers, acrylic resins, polyvinylpyrrolidone, cross-linked polyvinylpyrrolidone, polyethylene oxide, carbomer, diols, polyols, polyhydric alcohols, polyalkylene glycols, polyethylene glycols, polypropylene glycols or block polymers thereof, polyglycols, poly(α, Ω) alkylenediols; alkali metal salts and alkaline earth metal salts, and combinations thereof.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like.

Suitable plasticizers may include one or more of triacetin, diethyl phthalate, tributyl sebacate, polyethylene glycol and the like.

The pharmaceutical formulations as described herein may be prepared by processes known to the person having ordinary skill in the art of pharmaceutical technology such as direct compression, wet granulation, dry granulation or melt granulation.

The term “coat” as used herein is defined to mean a coating substantially surrounding a core which provides desirable properties to the dosage form. As is clear to the person of skill in the art, the coat can serve several purposes, including but not limited to protecting the dosage form from environmental conditions, such as light or moisture, providing esthetic or taste-masking properties to the dosage form, making the dosage form easier to swallow or to handle during the production process, or modifying the release properties of the dosage form, such that pharmaceutically active ingredient is released at a different rate from the coated core than from the uncoated core.

The sustained release formulation may be in the form of a tablet, a capsule, a caplet, a pouch, sprinkles, pellets, granules, or powder.

In one embodiment, the formulations may be prepared by mixing topiramate with one or more pharmaceutically acceptable excipients, lubricating and formulating into a suitable dosage form.

The pellets useful in the formulation of the present invention may comprise an inert carrier, topiramate, a binder, a controlled release agent and optionally, an overcoat that provides additional protection from moisture, static charge reduction, taste masking and coloring attributes to the particulates.

Topiramate is introduced to the inert carrier by techniques known to one skilled in the art, such as drug layering, powder coating, extrusion/spheronization, roller compaction or granulation. Preferably, the introduction method is drug layering by spraying a suspension of topiramate and a binder onto the inert carrier.

The current invention encompasses a method of preparing formulations of topiramate, comprising two extended release components or a combination of an extended release component and an immediate release component wherein topiramate is released from the formulation at the sustained rate along the pre-determined release profile.

In another embodiment, the method includes a process for providing an extended release component characterized by its own rate of release, wherein the process includes the steps of:
a) determining the desired release profile,
b) determining specific amounts of the extended release component and the immediate release component necessary to produce the pre- determined release profile; and
c) incorporating the specified amounts of the components into the formulation.

In one embodiment, the method comprises a step for providing an immediate release (IR) component, wherein the component may be prepared by sifting excipients followed by mixing with topiramate. The obtained mixture may be granulated with a solution of a binder to form granules. The granules may be dried and mixed with other pharmaceutically acceptable excipients, lubricated and compressed.

In another embodiment, the method comprises a step for providing an extended release component, wherein the component is contained in a population of pellets which may be prepared by the steps comprising:
(i) dispersing topiramate into a binder solution and/or other pharmaceutically acceptable excipients,
(ii) loading the inert carrier particles with topiramate dispersion,
(iii) drying, followed by optionally applying a barrier layer,
(iv) applying a controlled release coating,
(v) drying the coated pellets followed by a finish coating.

In another embodiment, the method for providing a matrix based extended release component, wherein the component may be prepared by the steps comprising:
(i) mixing excipients including a controlled release agent with topiramate,
(ii) granulating the obtained mixture with a solution of binder to form granules,
(iii) mixing the granules with other pharmaceutically acceptable excipients, lubricating and optionally compressing into a tablet.

The required quantities of two components/populations may then be filled in a Capsule.

Another embodiment discloses a method of treatment of a neurological and/or psychiatric condition, comprising orally administering to the subject a therapeutically effective amount of the sustained release formulation of the present invention, wherein said condition is selected from a group consisting of epilepsy, migraine, essential tremor, restless limb syndrome, cluster headaches, neuralgia, neuropathic pain, tourrette's syndrome, infantile spasms, bipolar disorder, dementia, depression, psychosis, mania, anxiety, schizophrenia, obsessive-compulsive disorder, post-traumatic stress disorder, attention deficit hyperactivity disorder, impulse control disorders, border line personality disorder, addiction, autism, chronic neurodegenerative disorders, acute neurodegeneration, amyotrophic lateral sclerosis.

In yet another embodiment, the sustained release formulation of topiramate exhibits no significant difference in both rate and extent of absorption of topiramate as compared to extended release formulation of topiramate marketed under the trade name TROKENDI®.

In yet another embodiment, the sustained release formulations provide for a mean Cₘₐₓ in the range of 80% to 125%, as compared to the currently marketed topiramate sustained release formulation “TROKENDI®”.

In yet another embodiment, the sustained release formulations provide for a mean AUC in the range of 80% to 125%, as compared to the currently marketed topiramate sustained release formulation “TROKENDI®”.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| | | |
|---|---|---|
| S.N | Ingredient | % w/w |

XR1 COMPONENT

| | | |
|---|---|---|
| 1 | Topiramate | 4-10 |
| 2 | Lactose Monohydrate | 20-35 |
| 3 | Hydroxypropyl methylcellulose | 2-10 |
| 4 | Povidone | 0.1-5 |
| 5 | Isopropyl Alcohol | q.s |
| 6 | Talc | 0.1-3 |
| 7 | Magnesium stearate | 0.1-5 |

XR2 COMPONENT
Drug loading

| | | |
|---|---|---|
| 1 | Microcrystalline cellulose NF Spheres | 25-35 |
| 2 | Topiramate USP | 12-20 |
| 3 | Povidone | 0.1-3 |
| 4 | Polyethylene glycol | 0.1-1 |
| 5 | Purified water | q.s |

BARRIER COATING

| | | |
|---|---|---|
| 6 | Opadry Clear | 1-4 |
| 7 | Purified water | q.s |

POLYMER COATING

| | | |
|---|---|---|
| 8 | Ethyl cellulose NF | 1-5 |
| 9 | Povidone | 0.5-2 |
| 10 | Triethyl citrate NF | 0.5-2 |
| 11 | Talc | 0.5-2 |
| 12 | Methylene chloride | q.s |
| 13 | Isopropyl alcohol | q.s |

FINISH COATING

| | | |
|---|---|---|
| 14 | Opadry AMB coating | 1-3 |
| 15 | Purified water | q.s |

### Process:

### XR1 Component:

Topiramate, lactose monohydrate and hydroxypropyl methylcellulose were sifted together and granulated using a binder solution comprising povidone. The granules were lubricated with magnesium stearate and compressed into tablets using suitable tooling.

### XR2 Component:

Topiramate was dispersed in a solution of povidone and polyethylene glycol followed by drug loading on inert microcrystalline cellulose spheres. The pellets were dried followed by a barrier coat application using the coating solution of opadry clear. The coated pellets were dried and further coated with controlled release coating solution comprising ethyl cellulose, povidone and triethyl citrate dissolved in a solution of methylene chloride and isopropyl alcohol. A finish coating was applied using opadry AMB.

Required quantities of two components/populations were filled in a Capsule.

### Example 2

TABLE 2

| | | |
|---|---|---|
| S.N | Ingredient | % w/w |

IR COMPONENT

| | | |
|---|---|---|
| 1 | Topiramate | 20-40 |
| 2 | Microcrystalline cellulose | 20-60 |
| 3 | Povidone | 3-10 |
| 4 | Purified water/Isopropyl Alcohol | q.s |
| 5 | Colloidal silicon dioxide | 1-3 |
| 6 | Magnesium stearate | 1-5 |

XR COMPONENT
Drug loading

| | | |
|---|---|---|
| 1 | Microcrystalline cellulose NF Spheres | 20-60 |
| 2 | Topiramate USP | 60-80 |
| 3 | Mannitol | 0.5-5 |
| 4 | Povidone | 1-5 |
| 5 | Polyethylene glycol | 0.1-5 |
| 6 | Purified water USP | q.s |

BARRIER COATING

| | | |
|---|---|---|
| 7 | Hydroxypropyl methylcellulose | 1-10 |
| 8 | Polyethylene glycol | 0.1-5 |
| 9 | Purified water USP | q.s |

POLYMER COATING

| | | |
|---|---|---|
| 10 | Ethyl cellulose | 2-10 |
| 11 | Povidone | 1-5 |
| 12 | Triethyl citrate NF | 0.1-5 |
| 13 | Dibutyl Sebecate | 0.1-5 |
| 14 | Talc | 1-5 |
| 15 | Methylene chloride | q.s |
| 16 | Isopropyl alcohol USP | q.s |

FINISH COATING

| | | |
|---|---|---|
| 17 | Opadry AMB coating | 1-4 |
| 18 | Purified water USP | q.s |

### Process:

### IR Component

Topiramate and microcrystalline cellulose were sifted together and granulated using a binder solution comprising povidone. The granules were lubricated with colloidal silicon dioxide and magnesium stearate and compressed into tablets using suitable tooling.

### XR Component

Topiramate was dispersed in a solution of mannitol, povidone and polyethylene glycol (PEG) followed by drug loading on inert microcrystalline cellulose (MCC) spheres. The pellets were dried followed by a barrier coat application using the coating solution of Hydroxypropyl methylcellulose 2910 and polyethylene glycol (PEG). The coated pellets were dried and further coated with controlled release coating solution comprising ethyl cellulose, povidone, triethyl citrate and dibutyl sebecate dissolved in a solution of methylene chloride and isopropyl alcohol. A finish coating was applied using opadry AMB.

Required quantities of two components/populations were filled in a Capsule.

### Example 3

TABLE 3

| | | |
|---|---|---|
| S.N | Ingredient | % w/w |

XR1 COMPONENT

| | | |
|---|---|---|
| 1 | Topiramate | 5.09 |
| 2 | Microcrystalline cellulose | 33.03 |
| 3 | Hydroxypropyl methylcellulose (methocel K4M) | 0.48 |
| 4 | Hydrogenated castor oil | 4.89 |
| 5 | Ethyl cellulose | 4.89 |
| 6 | Purified water | q.s |
| 7 | Purified talc | 0.49 |

XR2 COMPONENT

| | | |
|---|---|---|
| 8 | Microcrystalline cellulose (MCC) spheres | 29.45 |
| 9 | Topiramate | 15.27 |
| 10 | Povidone K90 | 0.92 |
| 11 | Polyethylene glycol 400 NF | 0.18 |
| 12 | Purified water | q.s |

BARRIER COATING

| | | |
|---|---|---|
| 13 | Opadry clear | 1.38 |
| 14 | Purified water | q.s |

POLYMER COATING

| | | |
|---|---|---|
| 15 | Ethyl cellulose 10 cps | 2.04 |
| 16 | Povidone K-30 | 0.68 |
| 17 | Triethyl citrate | 0.38 |
| 18 | Talc micronized | 0.68 |
| 19 | Methylene chloride | q.s |
| 20 | Isopropyl alcohol | q.s |

LUBRICATION

| | | |
|---|---|---|
| 21 | Purified talc | 0.14 |

### Process:

### XR1 Component

Half quantity of Hydroxypropyl methylcellulose (HPMC) was dissolved in water to get clear solution. Topiramate, microcrystalline cellulose, hydrogenated castor oil, ethyl cellulose were sifted and were granulated using HPMC solution. The wet mass was extruded and spheronized to provide pellets. The pellets were dried in fluid bed dryer. Appropriate sized pellets were selected and lubricated using purified talc.

### XR2 Component

Topiramate, povidone and polyethylene glycol were dispersed in purified water and the obtained solution was coated onto MCC spheres to achieve target weight of the pellets. Opadry clear was dissolved in purified water and a barrier coat was applied to the coated MCC spheres. Ethyl cellulose, povidone and triethyl citrate were dissolved in a solution of methylene chloride and isopropyl alcohol. Talc was dispersed into the obtained solution and the resulting solution was coated onto the pellets. The pellets were dried and lubricated with talc.

Required quantities of two components/populations were filled in a Capsule.

The dissolution performance for the two XR components, as well as the final formulation was measured using a USP-I rotating basket apparatus. Release times were measured by placing the tablet in a small wire basket placed on the end of a rod spinning at 150 rpm. Aliquots were withdrawn from pH 6.8 Ammonium phosphate buffer+0.01% SLS up to 16 hr.

TABLE 3a
Dissolution performance for the two XR components Example 3.

% drug
release

| Medium | Time (Hr) | XR1 | XR2 |
|---|---|---|---|
| pH 6.8 | 1 | 42.1 | 23.9 |
| Ammonium | 3 | 80.5 | 61.1 |
| phosphate | 6 | 97.1 | 85.2 |
| buffer + 0.01% | 12 | — | 90.9 |
| SLS | 16 | — | 92.9 |

TABLE 3b
Dissolution performance for the final formulation of Example 3.

| Medium | Time (Hr) | % drug release |
|---|---|---|
| pH 6.8 | 1 | 28.6 |
| Ammonium | 3 | 67.3 |
| phosphate | 6 | 88.4 |
| buffer + 0.01% | 8 | 92 |
| SLS | 12 | 95 |
| | 16 | 97.1 |

Topiramate Extended Release Capsules of the invention were evaluated for In-vivo parameters in fed and fasting studies.

### Bioavailability Study

In-vivo study was conducted in healthy human volunteers to assess bioavailability of topiramate extended release capsules (Test—composition of the invention as per Example 3) and TROKENDI® (Reference—Marketed topiramate extended release capsules)

TABLE 3c
Summary of PK parameters of Reference and Test compositions
under Fasting condition

| | REFERENCE | TEST GEOMETRIC |
|---|---|---|
| PARAMETER | GEOMETRIC MEANS | MEANS |
| Cmax (ng/ml) | 1109.642 | 1076.874 |
| AUCₜ (ng * h/ml) | 70278.077 | 72178.518 |
| AUCᵢ (ng * h/ml) | 74760.274 | 76586.963 |

TABLE 3d
Summary of PK parameters of Reference and Test compositions
under Fed condition:

| | REFERENCE | TEST GEOMETRIC |
|---|---|---|
| PARAMETER | GEOMETRIC MEANS | MEANS |
| Cmax (ng/ml) | 896.497 | 945.826 |
| AUCₜ (ng * h/ml) | 62710.441 | 64182.455 |
| AUCᵢ (ng * h/ml) | 67110.474 | 68488.170 |

1. A sustained release formulation of topiramate comprising two extended release components, a first extended release component (XR1) and a second extended release component (XR2), wherein topiramate is present in both the components and at least one of the components is present in a matrix form.

2. The sustained release formulation of topiramate according to claim 1, wherein the XR1 component comprises up to 50% by wt. of the total amount of the topiramate in the formulation and the XR2 component comprises at least 50% by wt. of the total amount of the topiramate in the formulation.

3. The sustained release formulation of topiramate according to claim 1, wherein the XR1 component releases about 80% of the topiramate in vitro in less than or equal to about 3 hours and about 97% of the topiramate in vitro in less than or equal to about 6 hours.

4. The sustained release formulation of topiramate according to claim 1, wherein the XR2 component releases about 85% of the topiramate in vitro in less than or equal to about 6 hours and releases about 90% of the topiramate in vitro in less than or equal to about 12 hours.

5. The sustained release formulation of topiramate according to claim 1, wherein the formulation releases about 30% of topiramate in about less than or equal to about 1 hour, releases about 35 to about 75% of topiramate in less than or equal to about 3 hours, releases more than about 80% topiramate in less than or equal to about 12 hours.

6. The sustained release formulation of topiramate according to claim 1, wherein the formulation comprises one or more controlled release agents comprising wax, methylcellulose, ethylcellulose, hydroxypropyl cellulose, hydroxypropyl methylcellulose, cellulose acetate, cellulose acetate phthalate, polyvinyl alcohol, hydroxypropyl methylcellulose phthalate, polyacrylates, polymethacrylates or copolymers thereof.

7. The sustained release formulation according to claim 6, wherein the controlled release agent comprises ethyl cellulose and hydroxypropyl methylcellulose.

8. The sustained release formulation of topiramate according to claim 1 further comprises one or more pharmaceutically acceptable excipients comprising fillers, disintegrants, binders, lubricants, wetting agents, glidants, pore forming agents, and the like.

9. The sustained release formulation of topiramate according to claim 1, wherein the formulation is in the form of a tablet, a capsule, a caplet, a pouch, sprinkles, pellets, granules, or powder.

10. The sustained release formulation according to claim 9, wherein the formulation is in the form of a capsule.

11. The sustained release formulation of topiramate according to claim 1, wherein the formulation exhibits no significant difference in both rate and extent of absorption of topiramate as compared to extended release formulation of topiramate marketed under the trade name Trokendi®.

12. The sustained release formulation of topiramate according to claim 1, wherein the formulation provides a mean AUC of plasma topiramate in both fed and fasted states within 80% to 125% of a mean AUC of plasma topiramate provided by a topiramate extended release reference standard upon single dose administration to a population of human subjects.

13. The sustained release formulation of topiramate according to claim 1, wherein the formulation provides a mean Cₘₐₓ of plasma topiramate in both fed and fasted states within 80% to 125% of a mean Cₘₐₓ of plasma topiramate provided by a topiramate extended release reference standard upon single dose administration to a population of human subjects.

14. A sustained release formulation of topiramate comprising an immediate release component (IR) and an extended release component (XR), wherein topiramate is present in both the components and at least one of the components is in a matrix form.

15. The sustained release formulation of topiramate according to claim 14, wherein the IR component comprises up to 50% by wt. of the total amount of the topiramate in the formulation and the XR component comprises at least 50% by wt. of the total amount of the topiramate in the formulation.

16. The sustained release formulation of topiramate according to claim 1, wherein the extended release component is prepared by a process comprising:
(i) coating a first layer of topiramate on inert carrier particles;
(ii) coating the particles with a second layer comprising one or more controlled release agents to obtain pellets; and
(iii) optionally, employing a barrier layer between the first and the second layer.

17. The sustained release formulation of topiramate according to claim 1, wherein the extended release component is prepared by a process comprising:
(i) mixing and/or granulating topiramate, one or more controlled release agents and one or more pharmaceutically acceptable excipients to form a wet mass;
(ii) extruding the wet mass of step (i);
(iii) spheronizing the product of step (ii) to form pellets; and
(iv) processing the pellets to form the extended release component of the final formulation.

18. The sustained release formulation of topiramate according to claim 1, wherein the extended release component is prepared by a process comprising:
(i) mixing and/or granulating topiramate, one or more controlled release agents and one or more pharmaceutically acceptable excipients to form a wet mass; and
drying, lubricating and compressing the obtained mass to form the extended release component of the final formulation.

19. The sustained release formulation of topiramate according to claim 14, wherein the extended release component is prepared by a process comprising:
(iv) coating a first layer of topiramate on inert carrier particles;
(v) coating the particles with a second layer comprising one or more controlled release agents to obtain pellets; and
(vi) optionally, employing a barrier layer between the first and the second layer.

20. The sustained release formulation of topiramate according to claim 14, wherein the extended release component is prepared by a process comprising:
(v) mixing and/or granulating topiramate, one or more controlled release agents and one or more pharmaceutically acceptable excipients to form a wet mass;
(vi) extruding the wet mass of step (i);
(vii) spheronizing the product of step (ii) to form pellets; and
(viii) processing the pellets to form the extended release component of the final formulation.

21. The sustained release formulation of topiramate according to claim 14, wherein the extended release component is prepared by a process comprising:
(ii) mixing and/or granulating topiramate, one or more controlled release agents and one or more pharmaceutically acceptable excipients to form a wet mass; and
drying, lubricating and compressing the obtained mass to form the extended release component of the final formulation.
